(19) United States
(12) Patent Application Publication
(10) Pub. No.: US 2008/0193400 A1
(43) Pub. Date: Aug. 14, 2008

(54) C-Glycoside Compounds and Use Thereof for Depigmenting the Skin

(76) Inventors: Maria Dalko, Gif S/Yvette (FR); Alexandre Cavezza, Pavillon sous Bois (FR)

Correspondence Address: OBLON, SPIVAK, MCCLELLAND MAIER & NEUSTADT, P.C., 1940 DUKE STREET, ALEXANDRIA, VA, 22314, US

(21) Appl. No.: 11/914,482

(22) PCT Filed: May 29, 2006
(86) PCT No.: PCT/EP06/06338

§ 371 Date: Nov. 15, 2007

Related U.S. Application Data
(60) Provisional application No. 60/687,307, filed on Jun. 6, 2005.

(30) Foreign Application Priority Data
May 30, 2005 (FR) 0551413

(51) Int. Cl. A61K 8/73; C07H 15/203; A61Q 19/02

(52) U.S. Cl. 424/62; 536/4.1

(57) ABSTRACT

The invention relates to a composition for depigmenting the skin comprising a compound of formula (I): in which: —S represents a monosaccharide or a polysaccharide containing up to 20 sugar units; —the bond S—CH₂ being a C-glycoside bond; —X represents a hydrogen atom or a group chosen from —OR₁, and —NR₁R₂ or an =0 group; —Y represents a group chosen from —OR₃, —NR₁R₂ and —OSi (R₄)₃; —Z represents a fluorine atom or a group chosen from a linear C₁-C₆ alkyl group, a cyclic or branched C₃-C₆ alkyl group, —OR₁, —NR₁R₂, —COR₁, —CO2R₁, —CONR₁R₂ and —CR₃; —m is an integer ranging from 0 to 4; with —R₁ and R₂ denoting, independently of each other, a hydrogen atom or a linear C₁-C₆ alkyl group, a cyclic or branched C₃-C₆ alkyl group, aralkyl or phenyl; —R₃ denoting a hydrogen atom or a linear C₁-C₆ alkyl group, a cyclic or branched C₃-C₆ alkyl group, aralkyl or phenyl, or a group —SO₃H, —PO₃H₂ or —COR₁; —R₄ denoting a linear C₁-C₆ alkyl group, a cyclic or branched C₃-C₆ alkyl group or phenyl; and also the cosmetically acceptable salts thereof, solvates thereof such as hydrates, and isomers thereof.

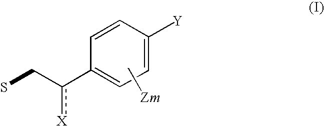

The present invention relates to a cosmetic composition for depigmenting and/or bleaching the skin, comprising a C-glycoside compound, and also to certain novel C-glycoside compounds.

The colour of human skin depends on different factors and, in particular, the seasons of the year, race and sex, and it is mainly determined by the nature and concentration of melanin produced by the melanocytes. Melanocytes are specialized cells which synthesize melanin by means of specific organelles, the melanosomes. In addition, at different periods in their life, certain individuals develop darker and/or more coloured blemishes on the skin and more especially on the hands, making the skin non-uniform. These blemishes are also due to a large concentration of melanin in the keratinocytes at the skin surface.

It is most particularly sought to use harmless topical depigmenting substances which are of good efficacy, in order to treat regional hyperpigmentations caused by melanocyte hyperactivity, such as idiopathic melasmas, occurring during pregnancy (“pregnancy mask” or chloasma) or during oestro-progestative contraception, localized hyperpigmentations caused by hyperactivity and proliferation of benign melanocytes, such as senile pigmentation marks known as actinic lentigo, accidental hyperpigmentations, possibly due to photosensitization or to post-lesional cicatrization, as well as certain leukodermias, such as vitiligo. For the latter, (in which the cicatrizations can result in a scar which gives the skin a whiter appearance), failing being able to repigment the damaged skin, the regions of residual normal skin are depigmented in order to give the skin as a whole a uniform white complexion.

The mechanism for the formation of skin pigmentation, that is to say the formation of melanin, is particularly complex and schematically involves the following main steps:

### Tyrosine -->Dopa -->Dopaquinone -->Dopachrome -->Melanin

Tyrosinase (monophenol dihydroxyl phenylalanine: oxygen oxidoreductase EC 1.14.18.1) is the essential enzyme involved in this reaction sequence. It especially catalyzes the reaction for the conversion of tyrosine into dopa (dihydroxyphenylalanine) by virtue of its hydroxylase activity and the reaction for the conversion of dopa into dopaquinone by virtue of its oxidase activity. This tyrosinase acts only when it is in the mature state, under the action of certain biological factors.

A substance is recognized as being depigmenting if it acts directly on the vitality of the epidermal melanocytes in which melanogenesis takes place, and/or if it interferes with one of the steps in the biosynthesis of melanin either by inhibiting one of the enzymes involved in melanogenesis or by becoming intercalated as a structural analogue of one of the chemical compounds in the melanin synthesis chain, whereby this chain may then be blocked and thus ensure the depigmentation.

The substances most commonly used as depigmenting agents are, more particularly, hydroquinone and its derivatives, in particular its ethers such as hydroquinone monomethyl ether and monoethyl ether. Although they have a certain level of efficacy, these compounds are unfortunately not free of side effects on account of their toxicity, which can make them difficult or even hazardous to use. This toxicity arises from the fact that they interfere with fundamental mechanisms of melanogenesis, by killing cells which then risk disrupting their biological environment and which consequently force the skin to eliminate them by producing toxins.

Thus, hydroquinone is a compound which is particularly irritant and cytotoxic to melanocytes, and whose total or partial replacement has been envisaged by many authors.

Substances have thus been sought which are not involved in the mechanism of melanogenesis, but which act upstream on tyrosinase by preventing its activation, and are consequently much less toxic. Kojic acid is commonly used as tyrosinase-activation inhibitor, this acid complexing the copper present in the active site of this enzyme. Unfortunately, this compound is unstable in solution, which somewhat complicates the manufacture of the composition.

There is still a need for a novel agent for bleaching human skin, which is just as effective as the known agents, but which does not have their drawbacks, i.e. which is non-irritant, non-toxic and/or non-allergizing for the skin, while at the same time being stable in a composition, or alternatively which has reinforced action so as to be able to be used in a smaller amount, thus considerably reducing the observed side effects.

In this regard, the Applicant has discovered, surprisingly and unexpectedly, that certain C-glycoside compounds have good depigmenting activity, even at low concentration, without showing any cytotoxicity.

One subject of the invention is, more specifically, a composition intended especially for depigmenting and/or bleaching the skin, comprising, in a physiologically acceptable medium, at least one compound of formula (I) below:

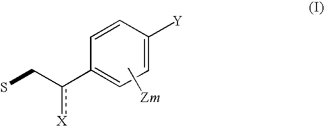

in which:
S represents a monosaccharide or a polysaccharide containing up to 20 sugar units, in pyranose and/or furanose form and of L and/or D series, the said monosaccharide or polysaccharide containing at least one hydroxyl function that is necessarily free and/or optionally one or more optionally protected amine functions;
the bond S—CH₂ being a C-glycoside bond;
X represents a hydrogen atom or a group chosen from —OR₁ and —NR₁R₂ or an ═O group;
Y represents a group chosen from —OR₃, —NR₁R₂ and —OSi (R₄)₃;
Z represents a fluorine atom or a group chosen from a linear C₁-C₆ alkyl group, a cyclic or branched C₃-C₆ alkyl group, —OR₁, —NR₁R₂, —COR₁, —CO₂R₁, —CONR₁R₂ and —CR₃;
m is an integer ranging from 0 to 4;
   with
R₁ and R₂ denoting, independently of each other, a hydrogen atom or a linear C₁-C₆ alkyl group, a cyclic or branched C₃-C₆ alkyl group, aralkyl or phenyl;
R₃ denoting a hydrogen atom or a linear C₁-C₆ alkyl group, a cyclic or branched C₃-C₆ alkyl group, aralkyl or phenyl, or a group —SO₃H, —PO₃H₂ or —COR₁;
R₄ denoting a linear C₁-C₆ alkyl group, a cyclic or branched C₃-C₆ alkyl group or phenyl;
   and also the cosmetically acceptable salts thereof, solvates thereof such as hydrates, and isomers thereof.

The compounds of formula (I) according to the invention allow the efficient depigmentation and/or lightening of human skin. They are especially intended to be applied to the skin of individuals with brownish pigmentation marks or senescence marks, or on the skin of individuals wishing to combat the appearance of a brownish colour originating from melanogenesis, for example after an exposure to ultraviolet radiation.

A subject of the invention is thus also a cosmetic process for depigmenting and/or bleaching human skin, comprising the application to the skin of a composition as described above. The process is especially suitable for eliminating brownish pigmentary marks and/or senescence marks, and/or for lightening browned skin.

A subject of the invention is also the cosmetic use of a compound of formula (I) as described above as an agent for bleaching and/or depigmenting the skin, especially for removing pigmentary marks and senescence marks and/or as anti-browning agents.

A subject of the invention is also the use of a compound of formula (I) as described above for the manufacture of a dermatological composition for depigmenting and/or bleaching the skin.

Among the compounds of formula (I), some are known in the prior art.

The document “Separation of α,β-anomers of C-glycosides by medium-pressure liquid chromatography”; September 1988, 6(5), pages 301-3 describes the compounds 2-deoxy-2-(2-(4-methoxyphenyl)ethyl)-D-glucose (CAS 121285-89-0) and 2-deoxy-2-(2-(4-methoxyphenyl)ethyl)-L-glucose (CAS 121285-90-3).

The document “Reaction of unsubstituted aldoses with p-methoxybenzoylmethylenephosphorane”; Zhurnal Obshchei Khimii 1968, 38(5), pages 1046-8 describes the compounds 2-deoxy-2-(2-oxo-2-(4-methoxyphenyl)ethyl)-L-fucose (CAS 20880-40-4), 2-deoxy-2-(2-oxo-2-(4-methoxyphenyl)ethyl)-D-xylose (CAS 20869-22-1) and 2-deoxy-2-(2-oxo-2-(4-methoxyphenyl)ethyl)-L-xylose (CAS 20869-24-3).

The document “Interaction of partially screened aldoses with p-methoxybenzoylmethylenephosphorane”; Zhurnal Obshchei Khimii 1969, 39(1), pages 119-22 describes the compounds 2-deoxy-2-(2-oxo-2-(4-methoxyphenyl)ethyl)-D-maltose (CAS 24461-52-7) and 2-deoxy-2-(2-oxo-2-(4-methoxyphenyl)ethyl)-L-maltose (CAS 24461-51-6).

A subject of the invention is thus also the compounds of formula (I′) corresponding to the compounds of formula (I) described above, with the proviso that when m=0 and Y denotes a —OCH₃ group, S does not denote a D- (or L)-glucose or L-fucose or D- (or L)-xylose or D- (or L)-maltose residue.

In the context of the present invention, the term “alkyl” means a saturated or unsaturated hydrocarbon-based chain. Among the alkyl groups that are suitable for use in the invention, mention may be made especially of methyl, ethyl, isopropyl, n-propyl, n-butyl, t-butyl, isobutyl, sec-butyl, pentyl, n-hexyl, cyclopropyl, cyclopentyl, cyclohexyl and allyl groups.

The acceptable salts for the non-therapeutic use of the compounds described in the present invention include the conventional non-toxic salts of the said compounds, such as those formed from organic or mineral acids. Examples that may be mentioned include the salts of mineral acids, such as sulfuric acid, hydrochloric acid, hydrobromic acid, hydriodic acid, phosphoric acid and boric acid. Mention may also be made of organic acid salts, which may comprise one or more carboxylic, sulfonic or phosphonic acid groups. These may be linear, branched or cyclic aliphatic acids or alternatively aromatic acids. These acids may also comprise one or more heteroatoms chosen from O and N, for example in the form of hydroxyl groups. Mention may be made especially of propionic acid, acetic acid, terephthalic acid, citric acid and tartaric acid.

When the compound of formula (I) comprises an acid group, the acid group(s) may be neutralized with a mineral base, such as LiOH, NaOH, KOH, Ca(OH)₂, NH₄OH, Mg(OH)₂ or Zn(OH)₂, or with an organic base such as a primary, secondary or tertiary alkylamine, for example triethylamine or butylamine. This primary, secondary or tertiary alkylamine may comprise one or more nitrogen and/or oxygen atoms and may thus comprise, for example, one or more alcohol functions; mention may be made especially of 2-amino-2-methylpropanol, triethanol-amine, dimethylamino-2-propanol and 2-amino-2-(hydroxymethyl)-1,3-propanediol. Mention may also be made of lysine or 3-(dimethylamino)propylamine.

The solvates that are acceptable for the non-therapeutic use of the compounds described in the present invention include conventional solvates such as those formed during the last step in the preparation of the said compounds due to the presence of solvents. Examples that may be mentioned include the solvates due to the presence of water or linear or branched alcohols, for instance ethanol or isopropanol.

One advantageous aspect of the invention relates to the compounds of formula (I) for which S represents a monosaccharide or disaccharide, the hydroxyl function in position 3 of which is free.

Advantageously, the preferred monosaccharides are chosen from D-glucose, D-galactose, D-mannose, D-fucose, L-fucose, D-xylose, D-lyxose, D-glucuronic acid, D-galacturonic acid, D-iduronic acid, N-acetyl-D-glucosamine and N-acetyl-D-galactosamine; preferably D-glucose, D-xylose, D-galactose, D-fucose and L-fucose, and more preferentially D-glucose.

Another particular aspect of the invention relates to the compounds of formula (I) for which S represents a polysaccharide as defined above.

Advantageously, the preferred polysaccharides contain up to 6 sugar units and are chosen from D-maltose, D-lactose, D-cellobiose, D-maltotriose, a disaccharide combining a uronic acid chosen from D-iduronic acid, D-glucuronic acid and D-galacturonic acid with a hexosamine chosen from D-galactosamine, D-glucosamine, N-acetyl-D-galactosamine, N-acetyl-D-glucosamine, an oligosaccharide containing at least one xylose advantageously chosen from xylobiose, methyl-β-xylobioside, xylotriose, xylotetraose, xylopentaose and xylohexaose and preferentially xylobiose, which is composed of two xylose molecules linked via a 1-4 bond. As particularly preferred polysaccharides, mention may be made of D-maltose and D-lactose.

Among the preferred alkyl groups, mention may be made of methyl, ethyl, propyl, isopropyl, butyl, isobutyl, tert-butyl, sec-butyl, pentyl, n-hexyl, cyclopropyl, cyclopentyl, cyclohexyl and allyl groups, preferentially methyl, ethyl, isopropyl and tert-butyl groups, and more preferentially a methyl group.

The preferred aryl group is the phenyl group.

The preferred compounds of formulae (I) and (I′) are those for which:
X denotes a group chosen from ═O, —OH, —NH₂ and —NHCH₃;
Z denotes a group chosen from —NH₂, —CH₃, —OH and —OCH₃;
Y denotes a group —OR₃ as defined above;
m denotes an integer equal to 0, 1 or 2.

The compounds of formulae (I) and (I′) that are particularly preferred are those for which:
S denotes a saccharide residue chosen from glucose, mannose, xylose, fucose, galactose, maltose, lactose, rhamnose, lyxose, arabinose, N-acetylglucosamine and N-acetylgalactosamine; and preferably chosen from glucose, xylose, galactose and fucose; and preferentially glucose;
X denotes a group chosen from ═O, —OH, —NH₂ and —NHCH₃; and preferentially ═O or —OH;
Y denotes a group chosen from —OPO₃H₂, —OSO₃H, —OAc, —OH and —OCH₃; and more particularly —OH or —OCH₃;
m=0.

The compounds of formulae (I) and (I′) that are more particularly preferred are the following:

2-deoxy-2-(2-hydroxy-2-(4-hydroxyphenyl)ethyl)-D(or L)-
glucose (Example 2)
2-deoxy-2-(2-hydroxy-2-(4-hydroxyphenyl)ethyl)-D(or L)-
galactose
2-deoxy-2-(2-hydroxy-2-(4-hydroxyphenyl)ethyl)-D(or L)-
maltose
2-deoxy-2-(2-hydroxy-2-(4-hydroxyphenyl)ethyl)-D(or L)-
mannose
2-deoxy-2-(2-hydroxy-2-(4-hydroxyphenyl)ethyl)-D(or L)-
xylose
2-deoxy-2-(2-hydroxy-2-(4-hydroxyphenyl)ethyl)-D(or L)-
fucose
2-deoxy-2-(2-hydroxy-2-(4-hydroxyphenyl)ethyl)-D(or L)-
lactose
2-deoxy-2-(2-hydroxy-2-(4-hydroxyphenyl)ethyl)-D(or L)-
rhamnose
2-deoxy-2-(2-hydroxy-2-(4-methoxyphenyl)ethyl)-D(or L)-
glucose (Example 4)
2-deoxy-2-(2-hydroxy-2-(4-methoxyphenyl)ethyl)-D(or L)-
galactose
2-deoxy-2-(2-hydroxy-2-(4-methoxyphenyl)ethyl)-D(or L)-
maltose
2-deoxy-2-(2-hydroxy-2-(4-methoxyphenyl)ethyl)-D(or L)-
mannose
2-deoxy-2-(2-hydroxy-2-(4-methoxyphenyl)ethyl)-D(or L)-
xylose
2-deoxy-2-(2-hydroxy-2-(4-methoxyphenyl)ethyl)-D(or L)-
fucose
2-deoxy-2-(2-hydroxy-2-(4-methoxyphenyl)ethyl)-D(or L)-
lactose
2-deoxy-2-(2-hydroxy-2-(4-methoxyphenyl)ethyl)-D(or L)-
rhamnose
2-deoxy-2-(2-oxo-2-(4-hydroxyphenyl)ethyl)-D(or L)-
glucose (Example 1)
2-deoxy-2-(2-oxo-2-(4-hydroxyphenyl)ethyl)-D(or L)-
galactose
2-deoxy-2-(2-oxo-2-(4-hydroxyphenyl)ethyl)-D(or L)-
maltose
2-deoxy-2-(2-oxo-2-(4-hydroxyphenyl)ethyl)-D(or L)-
mannose
2-deoxy-2-(2-oxo-2-(4-hydroxyphenyl)ethyl)-D(or L)-
xylose
2-deoxy-2-(2-oxo-2-(4-hydroxyphenyl)ethyl)-D(or L)-
fucose
2-deoxy-2-(2-oxo-2-(4-hydroxyphenyl)ethyl)-D(or L)-
lactose
2-deoxy-2-(2-oxo-2-(4-hydroxyphenyl)ethyl)-D(or L)-
rhamnose
2-deoxy-2-(2-oxo-2-(4-methoxyphenyl)ethyl)-D(or L)-
glucose (Example 3)
2-deoxy-2-(2-oxo-2-(4-methoxyphenyl)ethyl)-D(or L)-
galactose
2-deoxy-2-(2-oxo-2-(4-methoxyphenyl)ethyl)-D(or L)-
maltose
2-deoxy-2-(2-oxo-2-(4-methoxyphenyl)ethyl)-D(or L)-
mannose
2-deoxy-2-(2-oxo-2-(4-methoxyphenyl)ethyl)-D(or L)-
xylose
2-deoxy-2-(2-oxo-2-(4-methoxyphenyl)ethyl)-D(or L)-
fucose
2-deoxy-2-(2-oxo-2-(4-methoxyphenyl)ethyl)-D(or L)-
lactose
2-deoxy-2-(2-oxo-2-(4-methoxyphenyl)ethyl)-D(or L)
rhamnose
2-deoxy-2-(2-hydroxy-2-(3-methoxy-4-hydroxyphenyl)-
ethyl)-D(or L)-glucose
2-deoxy-2-(2-hydroxy-2-(3-methoxy-4-hydroxyphenyl)-
ethyl)-D(or L)-galactose
2-deoxy-2-(2-hydroxy-2-(3-methoxy-4-hydroxyphenyl)-
ethyl)-D(or L)-maltose
2-deoxy-2-(2-hydroxy-2-(3-methoxy-4-hydroxyphenyl)-
ethyl)-D(or L)-mannose
2-deoxy-2-(2-hydroxy-2-(3-methoxy-4-hydroxyphenyl)-
ethyl)-D(or L)-xylose
2-deoxy-2-(2-hydroxy-2-(3-methoxy-4-hydroxyphenyl)-
ethyl)-D(or L)-fucose
2-deoxy-2-(2-hydroxy-2-(3-methoxy-4-hydroxyphenyl)-
ethyl)-D(or L)-lactose
2-deoxy-2-(2-hydroxy-2-(3-methoxy-4-hydroxyphenyl)-
ethyl)-D(or L)-rhamnose
2-deoxy-2-(2-(4-hydroxyphenyl)ethyl)-D(or L)-glucose
2-deoxy-2-(2-(4-hydroxyphenyl)ethyl)-D(or L)-galactose
2-deoxy-2-(2-(4-hydroxyphenyl)ethyl)-D(or L)-maltose
2-deoxy-2-(2-(4-hydroxyphenyl)ethyl)-D(or L)-mannose
2-deoxy-2-(2-(4-hydroxyphenyl)ethyl)-D(or L)-xylose
2-deoxy-2-(2-(4-hydroxyphenyl)ethyl)-D(or L)-fucose
2-deoxy-2-(2-(4-hydroxyphenyl)ethyl)-D(or L)-lactose
2-deoxy-2-(2-(4-hydroxyphenyl)ethyl)-D(or L)-rhamnose
2-deoxy-2-(2-methylamino-2-(4-hydroxyphenyl)ethyl)-D(or
L)-glucose
2-deoxy-2-(2-methylamino-2-(4-hydroxyphenyl)ethyl)-D(or
L)-galactose
2-deoxy-2-(2-methylamino-2-(4-hydroxyphenyl)ethyl)-D(or
L)-maltose
2-deoxy-2-(2-methylamino-2-(4-hydroxyphenyl)ethyl)-D(or
L)-mannose
2-deoxy-2-(2-methylamino-2-(4-hydroxyphenyl)ethyl)-D(or
L)-xylose
2-deoxy-2-(2-methylamino-2-(4-hydroxyphenyl)ethyl)-D(or
L)-fucose
2-deoxy-2-(2-methylamino-2-(4-hydroxyphenyl)ethyl)-D(or
L)-lactose
2-deoxy-2-(2-methylamino-2-(4-hydroxyphenyl)ethyl)-D(or
L)-rhamnose
2-deoxy-2-(2-amino-2-(4-hydroxyphenyl)ethyl)-D(or L)-
glucose
2-deoxy-2-(2-amino-2-(4-hydroxyphenyl)ethyl)-D(or L)-
galactose
2-deoxy-2-(2-amino-2-(4-hydroxyphenyl)ethyl)-D(or L)-
maltose
2-deoxy-2-(2-amino-2-(4-hydroxyphenyl)ethyl)-D(or L)-
mannose
2-deoxy-2-(2-amino-2-(4-hydroxyphenyl)ethyl)-D(or L)-
xylose
2-deoxy-2-(2-amino-2-(4-hydroxyphenyl)ethyl)-D(or L)-
fucose
2-deoxy-2-(2-amino-2-(4-hydroxyphenyl)ethyl)-D(or L)-
lactose
2-deoxy-2-(2-amino-2-(4-hydroxyphenyl)ethyl)-D(or L)-
rhamnose
2-deoxy-2-(2-hydroxy-2-(4-hydroxyphenyl)ethyl)-D(or L)-
lyxose
2-deoxy-2-(2-hydroxy-2-(4-methoxyphenyl)ethyl)-D(or L)-
lyxose
2-deoxy-2-(2-oxo-2-(4-hydroxyphenyl)ethyl)-D(or L)-
lyxose
2-deoxy-2-(2-oxo-2-(4-methoxyphenyl)ethyl)-D(or L)-
lyxose
2-deoxy-2-(2-hydroxy-2-(3-methoxy-4-hydroxyphenyl)-
ethyl)-D(or L)-lyxose
2-deoxy-2-(2-(4-hydroxyphenyl)ethyl)-D(or L)-lyxose
2-deoxy-2-(2-methylamino-2-(4-hydroxyphenyl)ethyl)-D(or
L)-lyxose
2-deoxy-2-(2-amino-2-(4-hydroxyphenyl)ethyl)-D(or L)-
lyxose
2-deoxy-2-(2-hydroxy-2-(4-hydroxyphenyl)ethyl)-D(or L)-
arabinose
2-deoxy-2-(2-hydroxy-2-(4-methoxyphenyl)ethyl)-D(or L)-
arabinose
2-deoxy-2-(2-oxo-2-(4-hydroxyphenyl)ethyl)-D(or L)-
arabinose
2-deoxy-2-(2-oxo-2-(4-methoxyphenyl)ethyl)-D(or L)-
arabinose
2-deoxy-2-(2-hydroxy-2-(3-methoxy-4-hydroxyphenyl)-
ethyl)-D(or L)-arabinose
2-deoxy-2-(2-(4-hydroxyphenyl)ethyl)-D(or L)-arabinose
2-deoxy-2-(2-methylamino-2-(4-hydroxyphenyl)ethyl)-D(or
L)-arabinose
2-deoxy-2-(2-amino-2-(4-hydroxyphenyl)ethyl)-D(or L)-
arabinose
2-deoxy-2-(2-hydroxy-2-(4-hydroxyphenyl)ethyl)-D(or L)-
N-acetylglucosamine
2-deoxy-2-(2-hydroxy-2-(4-methoxyphenyl)ethyl)-D(or L)-
N-acetylglucosamine
2-deoxy-2-(2-oxo-2-(4-hydroxyphenyl)ethyl)-D(or L)-N-
acetylglucosamine
2-deoxy-2-(2-oxo-2-(4-methoxyphenyl)ethyl)-D(or L)-N-
acetylglucosamine
2-deoxy-2-(2-hydroxy-2-(3-methoxy-4-hydroxyphenyl)-
ethyl)-D(or L)-N-acetylglucosamine
2-deoxy-2-(2-(4-hydroxyphenyl)ethyl)-D(or L)-N-
acetylglucosamine
2-deoxy-2-(2-methylamino-2-(4-hydroxyphenyl)ethyl)-D(or
L)-N-acetylglucosamine
2-deoxy-2-(2-amino-2-(4-hydroxyphenyl)ethyl)-D(or L)-N-
acetylglucosamine
2-deoxy-2-(2-hydroxy-2-(4-hydroxyphenyl)ethyl)-D(or L)-
N-acetylgalactosamine
2-deoxy-2-(2-hydroxy-2-(4-methoxyphenyl)ethyl)-D(or L)-
N-acetylgalactosamine
2-deoxy-2-(2-oxo-2-(4-hydroxyphenyl)ethyl)-D(or L)-N-
acetylgalactosamine
2-deoxy-2-(2-oxo-2-(4-methoxyphenyl)ethyl)-D(or L)-N-
acetylgalactosamine
2-deoxy-2-(2-hydroxy-2-(3-methoxy-4-hydroxyphenyl)-
ethyl)-D(or L)-N-acetylgalactosamine
2-deoxy-2-(2-(4-hydroxyphenyl)ethyl)-D(or L)-N-
acetylgalactosamine
2-deoxy-2-(2-methylamino-2-(4-hydroxyphenyl)ethyl)-D(or
L)-N-acetylgalactosamine
2-deoxy-2-(2-amino-2-(4-hydroxyphenyl)ethyl)-D(or L)-N-
acetylgalactosamine

The compounds (I) and (I′) that are particularly preferred are chosen from:
2-deoxy-2-(2-hydroxy-2-(4-hydroxyphenyl)ethyl)-D(or L)-glucose
2-deoxy-2-(2-hydroxy-2-(4-methoxyphenyl)ethyl)-D(or L)-glucose
2-deoxy-2-(2-oxo-2-(4-methoxyphenyl)ethyl)-D(or L)-glucose
2-deoxy-2-(2-oxo-2-(4-hydroxyphenyl)ethyl)-D(or L)-glucose.

According to a first particular synthetic mode (Scheme I), the compounds of formula (I) for which C—X═C═O may be prepared by reacting a glycoside (II) with a diketo compound (III) in the presence of a base, at a temperature especially ranging from 70° C. to 100° C. The reaction may be performed in water or in a water/alcohol medium (such as ethanol, isopropanol or methanol). After cooling, the organic solvents possibly present are evaporated off and the residual aqueous phase is then diluted with water, then passed through an acidic resin and subsequently concentrated to isolate the compound of formula (Ia) obtained.

Bases that may be used include a mineral base such as sodium hydroxide, potassium hydroxide, sodium bicarbonate, potassium bicarbonate, sodium carbonate or potassium carbonate.

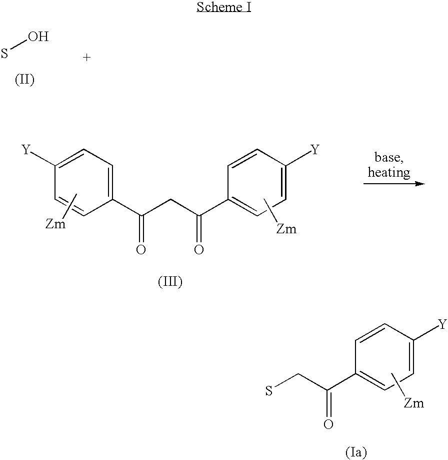

The diketo compound (III) may be prepared according to the synthetic scheme Ia, by reacting an ester (IIIa) (the alkyl group especially being a C₁-C₄ alkyl and in particular methyl or ethyl) and a ketone (IIIb) deprotonated beforehand with a strong base (for instance sodium hydroxide or lithium diisopropylamide), especially at a temperature of between 20° C. and 100° C. The reaction may be performed in an ether solvent (for instance dioxane, tetrahydrofuran or diisopropyl ether) or in a polar solvent (for instance dimethylformamide).

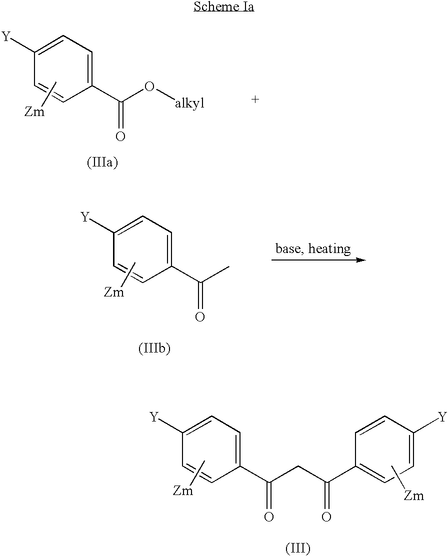

According to a second particular synthetic mode, the compounds of formula (I) for which C—X═C—OH may be prepared according to Scheme (II) via reduction of the keto compound (Ia) obtained according to the preparation mode described above (Scheme (I)) in the presence of a metal hydride such as sodium borohydride, lithium aluminium hydride or sodium bis(2-methoxyethoxy)-aluminium hydride (sold under the name Red-Al by the company Aldrich), especially in alcohol (such as ethanol, isopropanol or methanol) or an ether solvent (for instance dioxane, tetrahydrofuran or diisopropyl ether) at room temperature, for 1 to 20 hours. After reaction, the excess metal hydride is destroyed by adding acetone. After purification, compound (Ib) is obtained in the form of a mixture of diastereoisomers.

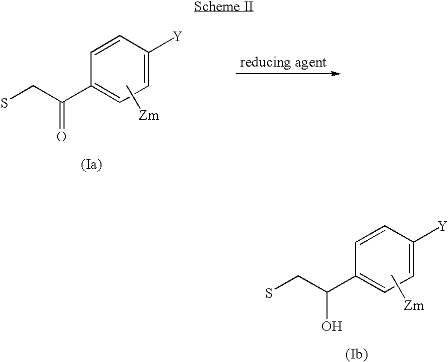

According to a third particular synthetic mode (Scheme III), the compounds of formula (I) for which C—X═C—NR₁R₂ may be prepared by reacting the keto compound (Ia) obtained according to the preparation mode described above (Scheme (I)) with an amine HNR₁R₂, especially in alcohol (such as ethanol or isopropanol) at a temperature of between 20° C. and 80° C., optionally in the presence of a drying agent such as magnesium sulfate, sodium sulfate or molecular sieves. The reaction is advantageously performed for 0.5 to 10 hours. A metal hydride (as indicated above) is then added portionwise, especially while allowing the reaction to proceed at room temperature for 1 to 20 hours. After reaction, the excess metal hydride is destroyed by adding acetone. After purification, compound (Ic) is obtained in the form of a mixture of diastereoisomers.

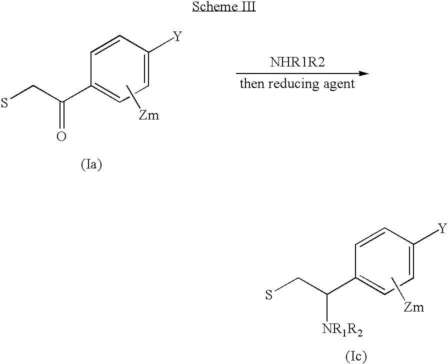

According to a fourth particular synthetic mode (Scheme IV), the compounds of formula (II) for which C—X═CH₂ may be prepared by reduction with activated zinc of the keto compound (Ia) obtained according to the preparation mode described above (Scheme (I)) and dissolved in acetic acid saturated with gaseous hydrogen chloride. The reaction is preferably first performed at about 0° C. and is then allowed to warm to room temperature and is left to react for about 10 hours. After purification, compound (Id) is obtained.

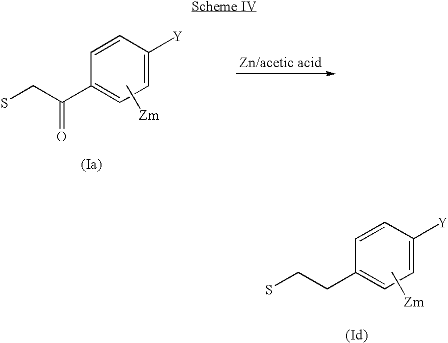

According to a fifth particular synthetic mode (Scheme V), the compounds of formula (I) for which C—X═C—OR₁ may be prepared according to the following sequence:

Step 1: Protection of the OH functions of the keto compound (Ia) obtained according to the preparation mode described above (Scheme (I)), in acetic anhydride in the presence of a catalytic amount of zinc chloride.

Step 2: Reduction according to a synthetic mode identical to that described for Scheme (II).

Step 3: Reaction of the intermediate compound with a strong base (for instance sodium hydride) and then with a compound R₁—X (X being a labile group such as halide, mesylate or tosylate), at a temperature of between 20° C. and 100° C. The reaction may be performed in an ether solvent (for instance dioxane, tetrahydrofuran or diisopropyl ether) or in an polar solvent (for instance dimethylformamide).

Step 4: Deprotection of the OAc functions with a mineral base (sodium hydroxide or lithium hydroxide) in a water/alcohol mixture (the alcohol possibly being methanol or ethanol) at a temperature of from 20° C. to 100° C., to obtain the compound of formula (Ie).

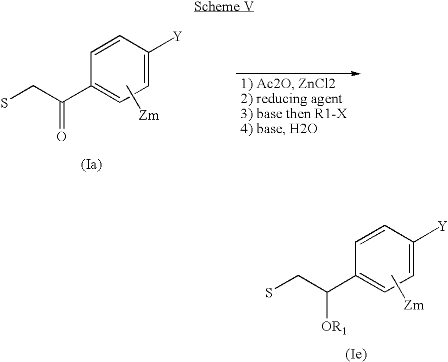

Another subject of the invention relates to a composition comprising, in a physiologically acceptable medium, at least one C-glycoside derivative corresponding to formula (I) as defined above. In particular, the composition is suitable for topical application to the skin. The physiologically acceptable medium will preferentially be a cosmetically or dermatologically acceptable medium, i.e. a medium with no unpleasant odour, colour or appearance, and which does not cause any stinging, tautness or redness that is unacceptable to the user.

The term “physiologically acceptable medium” means a medium that is compatible with human keratin materials, for instance the skin, mucous membranes, the nails, the scalp and/or the hair.

The composition according to the invention may be intended for cosmetic or pharmaceutical application, particularly dermatological application. The composition according to the invention is preferably intended for cosmetic application.

The amount of compounds of formula (I) that may be used in the context of the invention obviously depends on the desired effect.

By way of example, this amount may range, for example, from 0.001% to 10% by weight, preferably from 0.01% to 5% by weight and especially from 0.1% to 2% by weight relative to the total weight of the composition.

The composition may then comprise any constituent usually used in the intended application.

Mention may be made especially of water, solvents, oils of mineral, animal and/or plant origin, waxes, pigments, fillers, surfactants, cosmetic or dermatological active agents, UV-screening agents, polymers, gelling agents and preserving agents.

Needless to say, a person skilled in the art will take care to select this or these optional additional compound(s), and/or the amount thereof, such that the advantageous properties of the compounds according to the invention are not, or are not substantially, adversely affected by the envisaged addition.

The composition according to the invention may be in any pharmaceutical form normally used in cosmetics and dermatology, in particular in the form of an optionally gelled aqueous or aqueous-alcoholic solution, an optionally two-phase lotion-type dispersion, an oil-in-water or water-in-oil or multiple emulsion, an aqueous gel, a dispersion of oil in an aqueous phase with the aid of spherules, these spherules possibly being polymeric nanoparticles such as nanospheres and nanocapsules or better still lipid vesicles of ionic and/or non-ionic type.

When the composition of the invention is an emulsion, the proportion of the fatty phase can range from 5 to 80% by weight, and preferably from 5 to 50% by weight, relative to the total weight of the composition. The oils, the emulsifiers and the optional co-emulsifiers used in the composition in emulsion form are chosen from those used conventionally in the field considered. The emulsifier and the co-emulsifier are present in the composition in a proportion ranging from 0.3 to 30% by weight, and preferably from 0.5 to 20% by weight, relative to the total weight of the composition.

This composition may be relatively fluid and have the appearance of a white or coloured cream, an ointment, a milk, a lotion, a serum, a paste or a foam. It may optionally be applied to the skin in aerosol form. It may also be in solid form and, for example, in the form of a stick. It can be used as a care product and/or as a make-up product.

This composition may constitute a cleansing, protective, treatment or care cream for the face, the hands, the feet, the major anatomical folds or the body (for example day creams, night creams, makeup-removing creams, foundation creams or antisun creams); a fluid foundation, a makeup-removing milk, a protective or care body milk or an antisun milk; or a skincare lotion, gel or mousse, such as a cleansing lotion.

In one advantageous aspect of the invention, the compositions used may also comprise at least one desquamating agent, and/or at least one calmative and/or at least one organic photoprotective agent and/or at least one mineral photoprotective agent.

The term “desquamating agent” means any compound capable of acting:
either directly on the desquamation by promoting exfoliation, such as β-hydroxy acids, in particular salicylic acid and its derivatives (including 5-n-octanoylsalicylic acid); α-hydroxy acids, such as glycolic acid, citric acid, lactic acid, tartaric acid, malic acid or mandelic acid; urea; gentisic acid; oligofucoses; cinnamic acid; extract of Saphora japonica; resveratrol;
or on the enzymes involved in the desquamation or degradation of corneodesmosomes, glycosidases, stratum corneum chymotryptic enzyme (SCCE), or even other proteases (trypsin, chymotrypsin-like). Mention may be made of agents for chelating mineral salts: EDTA; N-acyl-N,N′,N′-ethylenediaminetriacetic acid; amino-sulfonic compounds and in particular (N-2-hydroxy-ethylpiperazine-N-2-ethane)sulfonic acid (HEPES); derivatives of 2-oxothiazolidine-4-carboxylic acid (procysteine); derivatives of α-amino acids of the type such as glycine (as described in EP-0 852 949, and sodium methylglycinediacetate sold by BASF under the trade name Trilon M); honey; sugar derivatives such as O-octanoyl-6-D-maltose and N-acetylglucosamine.

As calmatives that may be used in the composition according to the invention, mention may be made of: pentacyclic triterpenes and extracts of plants (e.g.: Glycyrrhiza glabra) containing them, for instance β-glycyrrhetinic acid and salts and/or derivatives thereof (glycyrrhetinic acid monoglucoronide, stearyl glycyrrhetinate or 3-stearoyloxyglycyrrhetic acid), ursolic acid and its salts, oleanolic acid and its salts, betulinic acid and its salts, an extract of Paeonia suffruticosa and/or lactiflora, salicylic acid salts and in particular zinc salicylate, the phycosaccharides from the company Codif, an extract of Laminaria saccharina, canola oil, bisabolol and camomile extracts, allantoin, Sepivital EPC (phosphoric diester of vitamins E and C) from SEPPIC, omega-3 unsaturated oils such as musk rose oil, blackcurrant oil, ecchium oil, fish oil, plankton extracts, capryloylglycine, Seppicalm VG (sodium palmitoylproline and Nymphea alba) from SEPPIC, an extract of Pygeum, an extract of Boswellia serrata, an extract of Centipeda cunnighami, an extract of Helianthus annuus, an extract of Linum usitatissimum, tocotrienols, extracts of Cola nitida, piperonal, an extract of clove, an extract of Epilobium angustifolium, Aloe vera, an extract of Bacopa moniera, phytosterols, cortisone, hydrocortisone, indomethacin and betamethasone.

The organic photoprotective agents are chosen especially from anthranilates; cinnamic derivatives; dibenzoylmethane derivatives; salicylic derivatives, camphor derivatives; triazine derivatives such as those described in patent applications U.S. Pat. No. 4,367,390, EP 863 145, EP 517 104, EP 570 838, EP 796 851, EP 775 698, EP 878 469, EP 933 376, EP 507 691, EP 507 692, EP 790 243 and EP 944 624; benzophenone derivatives; β,β-diphenylacrylate derivatives; benzotriazole derivatives; benzalmalonate derivatives; benzimidazole derivatives; imidazolines; bis-benzazolyl derivatives as described in patents EP 669 323 and U.S. Pat. No. 2,463,264; p-aminobenzoic acid (PABA) derivatives; methylenebis(hydroxyphenylbenzotriazole) derivatives as described in patent applications U.S. Pat. No. 5,237,071, U.S. Pat. No. 5,166,355, GB 2 303 549, DE 197 26 184 and EP 893 119; and screening polymers and screening silicones such as those described especially in patent application WO 93/04665; dimers derived from α-alkyl-styrene, such as those described in patent application DE 198 55 649.

The mineral photoprotective agents are chosen from pigments or nanopigments (mean size of the primary particles: generally between 5 nm and 100 nm and preferably between 10 nm and 50 nm) of coated or uncoated metal oxides, for instance nanopigments of titanium oxide (amorphous or crystallized in rutile and/or anatase form), of iron oxide, of zinc oxide, of zirconium oxide or of cerium oxide, which are all UV photoprotective agents that are well known per se. Standard coating agents are moreover alumina and/or aluminium stearate. Such coated or uncoated metal oxide nanopigments are described in particular in patent applications EP 518 772 and EP 518 773.

The photoprotective agents are generally present in the composition according to the invention in proportions ranging from 0.1% to 20% by weight relative to the total weight of the composition, and preferably ranging from 0.2% to 15% by weight relative to the total weight of the composition.

The examples that follow illustrate the invention without limiting its scope. Depending on the case, the compounds are cited as the chemical names or as the CTFA names (International Cosmetic Ingredient Dictionary and Handbook).

### EXAMPLE 1

### Synthesis of 2-deoxy-2-(2-oxo-2-(4-hydroxyphenyl)ethyl)-D(or L)-glucose

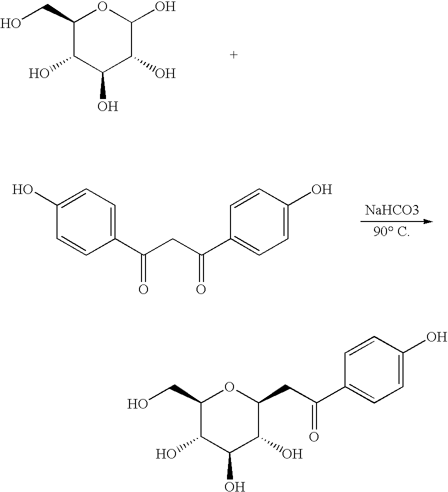

D-Glucose (60 mg, 0.33 mmol), water (0.25 mL) and sodium bicarbonate (NaHCO₃) were introduced into a round-bottomed flask, and 1,3-bis(4-hydroxyphenyl)-1,3-propanedione (127 mg, 0.5 mmol) dissolved in ethanol (0.75 mL) was then added. The mixture was heated at 90° C. for 20 hours. The medium was then cooled and the ethanol was evaporated off. The aqueous phase was diluted, extracted with dichloromethane, passed through Dowex® 50WX2-200 acid resin from Lancaster, and then concentrated. The residue (120 mg) obtained is a paste and is identified as being 2-deoxy-2-(2-oxo-2-(4-hydroxyphenyl)ethyl)-D(or L)-glucose.

The mass spectrometry is in accordance with the structure of the expected product.

### EXAMPLE 2

### Synthesis of 2-deoxy-2-(2-hydroxy-2-(4-hydroxyphenyl)-ethyl)-D(or L)-glucose

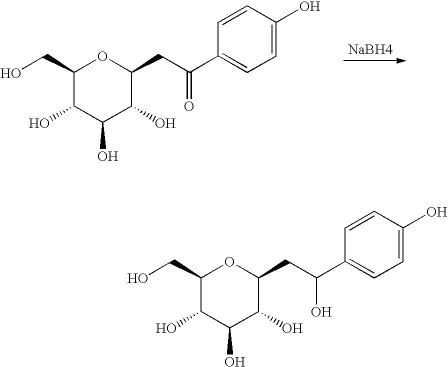

The crude product obtained in Example 1 (120 mg) was introduced into a round-bottomed flask and dissolved in methanol (5 mL). NaBH₄ (60 mg) was added portionwise and the mixture was then stirred at room temperature for 20 hours. Acetone (20 mL) was added to the medium. The medium was stirred for 1 hour and then concentrated. The residue was purified on a column of silica gel (for example silica gel 60 (0.040-0.063 mm from Merck) (eluent: 4/1 CH₂Cl₂/MeOH) to give 2-deoxy-2-(2-hydroxy-2-(4-hydroxyphenyl)ethyl)-D(or L)-glucose (80 mg) in the form of a paste containing 2 major diastereoisomers.

The analyses performed (¹H NMR; gas chromatography; mass spectrum) are in accordance with the expected structure of the product.

### EXAMPLE 3

### Synthesis of 2-deoxy-2-(2-oxo-2-(4-methoxyphenyl)ethyl)-D(or L)-glucose

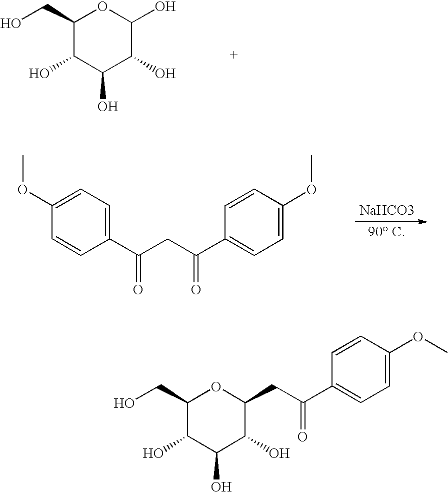

D-Glucose (2 g, 11.1 mmol), water (8 mL) and sodium bicarbonate (1.4 g, 16.7 mmol) were introduced into a round-bottomed flask, and 1,3-bis(4-methoxyphenyl)-1,3-propanedione (CAS 18362-51-1) (4.7 g, 16.7 mmol) dissolved in ethanol (25 mL) was then added. The mixture was heated at 90° C. for 20 hours. The medium was then cooled and the ethanol was evaporated off. The aqueous phase was diluted, extracted with dichloro-methane, passed through a Dowex® 50WX2-200 resin from Lancaster, and then concentrated. 1.8 g of 2-deoxy-2-(2-oxo-2-(4-methoxyphenyl)ethyl)-D(or L)-glucose were obtained.

The analyses performed (¹H NMR, mass spectrum) are in accordance with the expected structure.

### EXAMPLE 4

### Synthesis of 2-deoxy-2-(2-hydroxy-2-(4-methoxyphenyl)-ethyl)-D(or L)-glucose

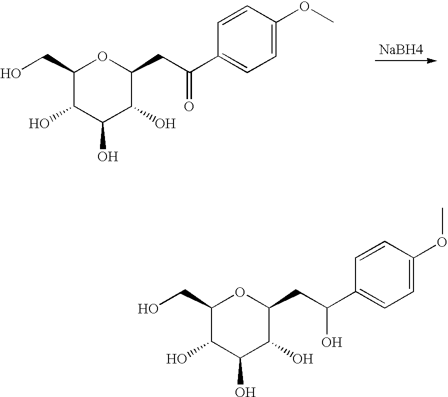

The product obtained in Example 3 (800 mg, 2.56 mmol) was introduced into a round-bottomed flask and dissolved in methanol (15 mL). NaBH₄ (114 mg, 3 mmol) was added portionwise and the mixture was then stirred at room temperature for 3 hours. Acetone was added to the medium, which was stirred for 1 hour and then concentrated. The residue obtained was purified on a column of silica gel (for example silica gel 60 (0.040-0.063 mm from Merck) (eluent: 4/1 CH₂Cl₂/MeOH), to give 2-deoxy-2-(2-hydroxy-2-(4-methoxyphenyl)ethyl)-D(or L)-glucose (798 mg) in the form of an oil containing a mixture of several diastereoisomers.

The analyses performed (¹H NMR, mass spectrum) are in accordance with the expected structure.

### EXAMPLE 5

### Demonstration of the Activity on Melanogenesis

A biological test demonstrated the depigmenting activity of the compound of Example 2.

The melanogenesis-modulating effect of the compound of Example 2 was measured according to the method described in patent FR-A-2 734 825, and also in the article by R. Schmidt, P. Krien and M. Régnier, Anal. Biochem., 235(2), 113-18, (1996). This test is performed on a co-culture of keratinocytes and melanocytes.

The following were determined for the test compound:
the cytotoxicity, by estimating the incorporation of leucine,
the inhibitory activity on melanin synthesis, by estimating the ratio of the incorporation of thiouracil to the incorporation of leucine, relative to 100% of the control (the control corresponds to the test performed without test compound). The IC₅₀ values (the concentration for which a 50% inhibition of melanogenesis is observed) were also determined.

The test was also performed with arbutin, which is a known depigmenting compound.

The results are collated in the following table:

| | Cytotoxicity | IC₅₀ (μM) |
|---|---|---|
| Compound of Ex. 2 | No | 100 |
| Arbutin | No | 100 |

The compound of Example 2 thus proves to be efficient in inhibiting melanogenesis; it is moreover more efficient than arbutin.

### EXAMPLE 6

A facial care bleaching cream of oil-in-water emulsion type is prepared, comprising (% by weight):

| | |
|---|---|
| compound of Example 1 | 0.005% |
| glyceryl stearate | 2% |
| Polysorbate 60 (Tween 60 from ICI) | 1% |
| stearic acid | 1.4% |
| triethanolamine | 0.7% |
| carbomer | 0.4% |
| liquid fraction of shea butter | 12% |
| perhydrosqualene | 12% |
| antioxidant | 0.05% |
| fragrance, preserving agent | qs |
| water | qs 100% |

A similar composition is prepared with the compound of Example 3.

### EXAMPLE 7

A skin depigmenting gel is prepared, comprising (% by weight):

| | |
|---|---|
| compound of Example 2 | 2% |
| hydroxypropylcellulose (Klucel H from | 1% |
| Hercules) | |
| antioxidant | 0.05% |
| isopropanol | 40% |
| fragrance, preserving agent | qs |
| water | qs 100% |

A similar composition is prepared with the compound of Example 4.

1. A composition comprising, in a physiologically acceptable medium, at least one compound of formula (I): 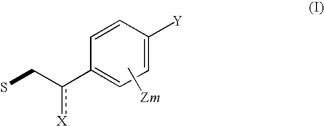
wherein:
S represents a monosaccharide or a polysaccharide containing up to 20 sugar units, in pyranose and/or furanose form and of L and/or D series, the said monosaccharide or polysaccharide containing at least one hydroxyl function that is necessarily free and/or optionally one or more optionally protected amine functions;
the bond S—CH₂ is a C-glycoside bond;
X represents a hydrogen atom, —OR₁, —NR₁R₂ or an ═O group;
Y represents —OR₃, —NR₁R₂ or —OSi(R₄)₃;
Z represents a fluorine atom or a group selected from the group consisting of a linear C₁-C₆ alkyl group, a cyclic or branched C₃-C₆ alkyl group, —OR₁, —NR₁R₂, —COR₁, —CO₂R₁, —CONR₁R₂ and —CR₃;
m is an integer ranging from 0 to 4;
R₁ and R₂ represent, independently of each other, a hydrogen atom, a linear C₁-C₆ alkyl group, a cyclic or branched C₃-C₆ alkyl group, aralkyl or phenyl;
R₃ represents a hydrogen atom, a linear C₁-C₆ alkyl group, a cyclic or branched C₃-C₆ alkyl group, aralkyl, phenyl, —SO₃H, —PO₃H₂ or —COR₁;
R₄ represents a linear C₁-C₆ alkyl group, a cyclic or branched C₃-C₆ alkyl group or phenyl;
and the cosmetically acceptable salts thereof, solvates thereof, and isomers thereof.

2. The composition according to claim 1, wherein S is a monosaccharide or disaccharide, comprising a free hydroxyl group in position 3.

3. The composition according to claim 1, wherein S represents a monosaccharide selected from the group consisting of D-glucose, D-galactose, D-mannose, D-fucose, L-fucose, D-xylose, D-lyxose, D-glucuronic acid, D-galacturonic acid, D-iduronic acid, N-acetyl-D-glucosamine and N-acetyl-D-galactosamine.

4. The composition according to claim 3, wherein S is D-glucose.

5. The composition according to claim 1, wherein S represents a polysaccharide comprising up to 6 sugar units.

6. The composition according to claim 1, wherein S represents a polysaccharide selected from the group consisting of D-maltose, D-lactose, D-cellobiose, D-maltotriose, a disaccharide combining a uronic acid selected from the group consisting of D-iduronic acid, D-glucuronic acid and D-galacturonic acid with a hexosamine selected from the group consisting of D-galactosamine, D-glucosamine, N-acetyl-D-galactosamine, N-acetyl-D-glucosamine, an oligosaccharide containing at least one xylose.

7. The composition according to claim 6, wherein S represents a polysaccharide selected from the group consisting of D-maltose and D-lactose.

8. The composition according to claim 1, wherein:
X denotes ═O, —OH, —NH₂ or —NHCH₃;
Z denotes —NH₂, —CH₃, —OH or —OCH₃;
Y denotes —OR₃ as defined in claim 1; and
m denotes an integer equal to 0, 1 or 2.

9. The composition according to claim 1, wherein:
S denotes a saccharide residue selected from the group consisting of glucose, mannose, xylose, fucose, galactose, maltose, lactose, rhamnose, lyxose, arabinose, N-acetylglucosamine and N-acetylgalactosamine;
X denotes ═O, —OH, —NH₂ or —NHCH₃;
Y denotes —OPO₃H₂, —OSO₃H, —OAc, —OH or —OCH₃; and
m=0.

10. The composition according to claim 1, wherein the compound of formula (I) is selected from the group consisting of:
2-deoxy-2-(2-hydroxy-2-(4-hydroxyphenyl)ethyl)-D(or L)-glucose
2-deoxy-2-(2-hydroxy-2-(4-hydroxyphenyl)ethyl)-D(or L)-galactose
2-deoxy-2-(2-hydroxy-2-(4-hydroxyphenyl)ethyl)-D(or L)-maltose
2-deoxy-2-(2-hydroxy-2-(4-hydroxyphenyl)ethyl)-D(or L)-mannose
2-deoxy-2-(2-hydroxy-2-(4-hydroxyphenyl)ethyl)-D(or L)-xylose
2-deoxy-2-(2-hydroxy-2-(4-hydroxyphenyl)ethyl)-D(or L)-fucose
2-deoxy-2-(2-hydroxy-2-(4-hydroxyphenyl)ethyl)-D(or L)-lactose
2-deoxy-2-(2-hydroxy-2-(4-hydroxyphenyl)ethyl)-D(or L)-rhamnose
2-deoxy-2-(2-hydroxy-2-(4-methoxyphenyl)ethyl)-D(or L)-glucose
2-deoxy-2-(2-hydroxy-2-(4-methoxyphenyl)ethyl)-D(or L)-galactose
2-deoxy-2-(2-hydroxy-2-(4-methoxyphenyl)ethyl)-D(or L)-maltose
2-deoxy-2-(2-hydroxy-2-(4-methoxyphenyl)ethyl)-D(or L)-mannose
2-deoxy-2-(2-hydroxy-2-(4-methoxyphenyl)ethyl)-D(or L)-xylose
2-deoxy-2-(2-hydroxy-2-(4-methoxyphenyl)ethyl)-D(or L)-fucose
2-deoxy-2-(2-hydroxy-2-(4-methoxyphenyl)ethyl)-D(or L)-lactose
2-deoxy-2-(2-hydroxy-2-(4-methoxyphenyl)ethyl)-D(or L)-rhamnose
2-deoxy-2-(2-oxo-2-(4-hydroxyphenyl)ethyl)-D(or L)-glucose
2-deoxy-2-(2-oxo-2-(4-hydroxyphenyl)ethyl)-D(or L)-galactose
2-deoxy-2-(2-oxo-2-(4-hydroxyphenyl)ethyl)-D(or L)-maltose
2-deoxy-2-(2-oxo-2-(4-hydroxyphenyl)ethyl)-D(or L)-mannose
2-deoxy-2-(2-oxo-2-(4-hydroxyphenyl)ethyl)-D(or L)-xylose
2-deoxy-2-(2-oxo-2-(4-hydroxyphenyl)ethyl)-D(or L)-fucose
2-deoxy-2-(2-oxo-2-(4-hydroxyphenyl)ethyl)-D(or L)-lactose
2-deoxy-2-(2-oxo-2-(4-hydroxyphenyl)ethyl)-D(or L)-rhamnose
2-deoxy-2-(2-oxo-2-(4-methoxyphenyl)ethyl)-D(or L)-glucose
2-deoxy-2-(2-oxo-2-(4-methoxyphenyl)ethyl)-D(or L)-galactose
2-deoxy-2-(2-oxo-2-(4-methoxyphenyl)ethyl)-D(or L)-maltose
2-deoxy-2-(2-oxo-2-(4-methoxyphenyl)ethyl)-D(or L)-marmose
2-deoxy-2-(2-oxo-2-(4-methoxyphenyl)ethyl)-D(or L)-xylose
2-deoxy-2-(2-oxo-2-(4-methoxyphenyl)ethyl)-D(or L)-fucose
2-deoxy-2-(2-oxo-2-(4-methoxyphenyl)ethyl)-D(or L)-lactose
2-deoxy-2-(2-oxo-2-(4-methoxyphenyl)ethyl)-D(or L)-rhamnose
2-deoxy-2-(2-hydroxy-2-(3-methoxy-4-hydroxyphenyl)ethyl)-D(or L)-glucose
2-deoxy-2-(2-hydroxy-2-(3-methoxy-4-hydroxyphenyl)ethyl)-D(or L)-galactose
2-deoxy-2-(2-hydroxy-2-(3-methoxy-4-hydroxyphenyl)ethyl)-D(or L)-maltose
2-deoxy-2-(2-hydroxy-2-(3-methoxy-4-hydroxyphenyl)ethyl)-D(or L)-mannose
2-deoxy-2-(2-hydroxy-2-(3-methoxy-4-hydroxyphenyl)ethyl)-D(or L)-xylose
2-deoxy-2-(2-hydroxy-2-(3-methoxy-4-hydroxyphenyl)ethyl)-D(or L)-fucose
2-deoxy-2-(2-hydroxy-2-(3-methoxy-4-hydroxyphenyl)ethyl)-D(or L)-lactose
2-deoxy-2-(2-hydroxy-2-(3-methoxy-4-hydroxyphenyl)ethyl)-D(or L)-rhamnose
2-deoxy-2-(2-(4-hydroxyphenyl)ethyl)-D(or L)-glucose
2-deoxy-2-(2-(4-hydroxyphenyl)ethyl)-D(or L)-galactose
2-deoxy-2-(2-(4-hydroxyphenyl)ethyl)-D(or L)-maltose
2-deoxy-2-(2-(4-hydroxyphenyl)ethyl)-D(or L)-mannose
2-deoxy-2-(2-(4-hydroxyphenyl)ethyl)-D(or L)-xylose
2-deoxy-2-(2-(4-hydroxyphenyl)ethyl)-D(or L)-fucose
2-deoxy-2-(2-(4-hydroxyphenyl)ethyl)-D(or L)-lactose
2-deoxy-2-(2-(4-hydroxyphenyl)ethyl)-D(or L)-rharnnose
2-deoxy-2-(2-methylamino-2-(4-hydroxyphenyl)ethyl)-D(or L)-glucose
2-deoxy-2-(2-methylamino-2-(4-hydroxyphenyl)ethyl)-D(or L)-galactose
2-deoxy-2-(2-methylamino-2-(4-hydroxyphenyl)ethyl)-D(or L)-maltose
2-deoxy-2-(2-methylamino-2-(4-hydroxyphenyl)ethyl)-D(or L)-mannose
2-deoxy-2-(2-methylamino-2-(4-hydroxyphenyl)ethyl)-D(or L)-xylose
2-deoxy-2-(2-methylamino-2-(4-hydroxyphenyl)ethyl)-D(or L)-fucose
2-deoxy-2-(2-methylamino-2-(4-hydroxyphenyl)ethyl)-D(or L)-lactose
2-deoxy-2-(2-methylamino-2-(4-hydroxyphenyl)ethyl)-D(or L)-rhamnose
2-deoxy-2-(2-amino-2-(4-hydroxyphenyl)ethyl)-D(or L)-glucose
2-deoxy-2-(2-amino-2-(4-hydroxyphenyl)ethyl)-D(or L)-galactose
2-deoxy-2-(2-amino-2-(4-hydroxyphenyl)ethyl)-D(or L)-maltose
2-deoxy-2-(2-amino-2-(4-hydroxyphenyl)ethyl)-D(or L)-mannose
2-deoxy-2-(2-amino-2-(4-hydroxyphenyl)ethyl)-D(or L)-xylose
2-deoxy-2-(2-amino-2-(4-hydroxyphenyl)ethyl)-D(or L)-fucose
2-deoxy-2-(2-amino-2-(4-hydroxyphenyl)ethyl)-D(or L)-lactose
2-deoxy-2-(2-amino-2-(4-hydroxyphenyl)ethyl)-D(or L)-rhamnose
2-deoxy-2-(2-hydroxy-2-(4-hydroxyphenyl)ethyl)-D(or L)-lyxose
2-deoxy-2-(2-hydroxy-2-(4-rnethoxyphenyl)ethyl)-D(or L)-lyxose
2-deoxy-2-(2-oxo-2-(4-hydroxyphenyl)ethyl)-D(or L)-lyxose
2-deoxy-2-(2-oxo-2-(4-methoxyphenyl)ethyl)-D(or L)-lyxose
2-deoxy-2-(2-hydroxy-2-(3-methoxy-4-hydroxyphenyl)ethyl)-D(or L)-lyxose
2-deoxy-2-(2-(4-hydroxyphenyl)ethyl)-D(or L)-lyxose
2-deoxy-2-(2-methylamino-2-(4-hydroxyphenyl)ethyl)-D(or L)-lyxose
2-deoxy-2-(2-amino-2-(4-hydroxyphenyl)ethyl)-D(or L)-lyxose
2-deoxy-2-(2-hydroxy-2-(4-hydroxyphenyl)ethyl)-D(or L)-arabinose
2-deoxy-2-(2-hydroxy-2-(4-methoxyphenyl)ethyl)-D(or L)-arabinose
2-deoxy-2-(2-oxo-2-(4-hydroxyphenyl)ethyl)-D(or L)-arabinose
2-deoxy-2-(2-oxo-2-(4-methoxyphenyl)ethyl)-D(or L)-arabinose
2-deoxy-2-(2-hydroxy-2-(3-methoxy-4-hydroxyphenyl)ethyl)-D(or L)-arabinose
2-deoxy-2-(2-(4-hydroxyphenyl)ethyl)-D(or L)-arabinose
2-deoxy-2-(2-methylamino-2-(4-hydroxyphenyl)ethyl)-D(or L)-arabinose
2-deoxy-2-(2-amino-2-(4-hydroxyphenyl)ethyl)-D(or L)-arabinose
2-deoxy-2-(2-hydroxy-2-(4-hydroxyphenyl)ethyl)-D(or L)-N-acetylglucosamine
2-deoxy-2-(2-hydroxy-2-(4-methoxyphenyl)ethyl)-D(or L)-N-acetylglucosamine
2-deoxy-2-(2-oxo-2-(4-hydroxyphenyl)ethyl)-D(or L)-N-acetylglucosamine
2-deoxy-2-(2-oxo-2-(4-methoxyphenyl)ethyl)-D(or L)-N-acetylglucosamine
2-deoxy-2-(2-hydroxy-2-(3-methoxy-4-hydroxyphenyl)ethyl)-D(or L)-N-acetylglucosamine
2-deoxy-2-(2-(4-hydroxyphenyl)ethyl)-D(or L)-N-acetylglucosamine
2-deoxy-2-(2-methylamino-2-(4-hydroxyphenyl)ethyl)-D(or L)-N-acetylglucosamine
2-deoxy-2-(2-amino-2-(4-hydroxyphenyl)ethyl)-D(or L)-N-acetylglucosarine
2-deoxy-2-(2-hydroxy-2-(4-hydroxyphenyl)ethyl)-D(or L)-N-acetylgalactosamine
2-deoxy-2-(2-hydroxy-2-(4-methoxyphenyl)ethyl)-D(or L)-N-acetylgalactosa ine
2-deoxy-2-(2-oxo-2-(4-hydroxyphenyl)ethyl)-D(or L)-N-acetylgalactosamine
2-deoxy-2-(2-oxo-2-(4-methoxyphenyl)ethyl)-D(or L)-N-acetylgalactosamine
2-deoxy-2-(2-hydroxy-2-(3-methoxy-4-hydroxyphenyl)ethyl)-D(or L)-N-acetylgalactosamine
2-deoxy-2-(2-(4-hydroxyphenyl)ethyl)-D(or L)-N-acetylgalactosamine
2-deoxy-2-(2-methylamino-2-(4-hydroxyphenyl)ethyl)-D(or L)-N-acetylgalactosamine
2-deoxy-2-(2-amino-2-(4-hydroxyphenyl)ethyl)-D(or L)-N-acetylgalactosamine.

11. The composition according to claim 10, wherein the compound of formula (I) is selected from the group consisting of:
2-deoxy-2-(2-oxo-2-(4-hydroxyphenyl)ethyl)-D(or L)-glucose
2-deoxy-2-(2-hydroxy-2-(4-hydroxyphenyl)ethyl)-D(or L)-glucose
2-deoxy-2-(2-oxo-2-(4-methoxyphenyl)ethyl)-D(or L)-glucose
2-deoxy-2-(2-hydroxy-2-(4-methoxyphenyl)ethyl)-D(or L)-glucose.

12. The composition according to claim 1, wherein the compound of formula (I) is present in an amount ranging from 0.001% to 10% by weight relative to the total weight of the composition.

13. A method for depigmenting and/or bleaching skin comprising treating the skin with the composition according to claim 1.

14. A cosmetic process for depigmenting and/or bleaching human skin, body hairs or head hair, comprising applying to the human skin, body hairs or head hair the composition according to claim 1.

15. A cosmetic agent for bleaching and/or depigmenting the skin comprising the composition according to claim 1.

16. A method for the manufacture of a dermatological composition for depigmenting and/or bleaching the skin comprising adding the composition according to claim 1.

17. A C-Glycoside compound of formula (I′): 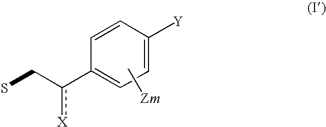
wherein:
S is a monosaccharide or a polysaccharide containing up to 20 sugar units, in pyranose and/or furanose form and of L and/or D series, the said monosaccharide or polysaccharide containing at least one hydroxyl function that is necessarily free and/or optionally one or more optionally protected amine functions;
the bond S—CH₂ is a C-glycoside bond;
X represents a hydrogen atom, —OR₁, —NR₁R₂ or an ═O group;
Y represents a —OR₃, —NR₁R₂ or —OSi(R₄)₃;
Z represents a fluorine atom or a group selected from the group consisting of a linear C₁-C₆ alkyl group, a cyclic or branched C₃-C₆ alkyl group, —OR₁, —NR₁R₂, —COR₁, —CO₂R₁, —CONR₁R₂ and —CR₃;
m is an integer ranging from 0 to 4;
R₁ and R₂ represent, independently of each other, a hydrogen atom, a linear C₁-C₆ alkyl group, a cyclic or branched C₃-C₆ alkyl group, aralkyl or phenyl;
R₃ represents a hydrogen atom, a linear C₁-C₆ alkyl group, a cyclic or branched C₃-C₆ alkyl group, aralkyl, phenyl, —SO₃H, —PO₃H₂ or —COR₁;
R₄ represents a linear C₁-C₆ alkyl group, a cyclic or branched C₃-C₆ alkyl group or phenyl;
with the proviso that when m=0 and Y denotes an —OCH₃ group, S does not denote a D- or L-glucose, L-fucose, D- or L-xylose, or D- or L-maltose residue,
and the cosmetically acceptable salts thereof, solvates thereof, and isomers thereof.

18. The C-Glycoside compound according to claim 17, wherein S represents a monosaccharide or disaccharide, comprising a free hydroxyl group in position 3.

19. The C-Glycoside compound according to claim 17, wherein S is a monosaccharide selected from the group consisting of D-glucose, D-galactose, D-mannose, D-fucose, L-fucose, D-xylose, D-lyxose, D-glucuronic acid, D-galacturonic acid, D-iduronic acid, N-acetyl-D-glucosamine and N-acetyl-D-galactosamine.

20. The C-Glycoside compound according to claim 19, wherein S is D-glucose.

21. The C-Glycoside compound according to claim 17, wherein S is a polysaccharide containing up to 6 sugar units.

22. The C-Glycoside compound according to claim 21, wherein S is a polysaccharide selected from the group consisting of D-maltose, D-lactose, D-cellobiose, D-maltotriose, a disaccharide combining a uronic acid selected from the group consisting of D-iduronic acid, D-glucuronic acid and D-galacturonic acid with a hexosamine selected from the group consisting of D-galactosamine, D-glucosamine, N-acetyl-D-galactosamine, N-acetyl-D-glucosamine, and an oligosaccharide containing at least one xylose.

23. The C-Glycoside compound according to claim 21, wherein is D-maltose or D-lactose.

24. The C-Glycoside compound according to claim 17, wherein:
X is ═O, —OH, —NH₂ or —NHCH₃;
Z is —NH₂, —CH₃, —OH or —OCH₃;
Y is —OR₃ as defined in claim 17;
m is an integer equal to 0, 1 or 2.

25. The C-Glycoside compound according to claim 17, wherein:
S is a saccharide residue selected from the group consisting of glucose, mannose, xylose, fucose, galactose, maltose, lactose, rhamnose, lyxose, arabinose, N-acetylglucosamine and N-acetylgalactosamine;
X is ═O, —OH, —NH₂ or —NHCH₃;
Y is —OPO₃H₂, —OSO₃H, —OAc, —OH or —OCH₃;
m=0.

26. The C-Glycoside compound according to claim 17, wherein the compound of formula (I′) is selected from the group consisting of:
2-deoxy-2-(2-hydroxy-2-(4-hydroxyphenyl)ethyl)-D(or L)-glucose
2-deoxy-2-(2-hydroxy-2-(4-hydroxyphenyl)ethyl)-D(or L)-galactose
2-deoxy-2-(2-hydroxy-2-(4-hydroxyphenyl)ethyl)-D(or L)-maltose
2-deoxy-2-(2-hydroxy-2-(4-hydroxyphenyl)ethyl)-D(or L)-mannose
2-deoxy-2-(2-hydroxy-2-(4-hydroxyphenyl)ethyl)-D(or L)-xylose
2-deoxy-2-(2-hydroxy-2-(4-hydroxyphenyl)ethyl)-D(or L)-fucose
2-deoxy-2-(2-hydroxy-2-(4-hydroxyphenyl)ethyl)-D(or L)-lactose
2-deoxy-2-(2-hydroxy-2-(4-hydroxyphenyl)ethyl)-D(or L)-rhamnose
2-deoxy-2-(2-hydroxy-2-(4-methoxyphenyl)ethyl)-D(or L)-glucose
2-deoxy-2-(2-hydroxy-2-(4-methoxyphenyl)ethyl)-D(or L)-galactose
2-deoxy-2-(2-hydroxy-2-(4-methoxyphenyl)ethyl)-D(or L)-maltose
2-deoxy-2-(2-hydroxy-2-(4-methoxyphenyl)ethyl)-D(or L)-mannose
2-deoxy-2-(2-hydroxy-2-(4-methoxyphenyl)ethyl)-D(or L)-xylose
2-deoxy-2-(2-hydroxy-2-(4-methoxyphenyl)ethyl)-D(or L)-fucose
2-deoxy-2-(2-hydroxy-2-(4-methoxyphenyl)ethyl)-D(or L)-lactose
2-deoxy-2-(2-hydroxy-2-(4-methoxyphenyl)ethyl)-D(or L)-rhamnose
2-deoxy-2-(2-oxo-2-(4-hydroxyphenyl)ethyl)-D(or L)-glucose
2-deoxy-2-(2-oxo-2-(4-hydroxyphenyl)ethyl)-D(or L)-galactose
2-deoxy-2-(2-oxo-2-(4-hydroxyphenyl)ethyl)-D(or L)-maltose
2-deoxy-2-(2-oxo-2-(4-hydroxyphenyl)ethyl)-D(or L)-mannose
2-deoxy-2-(2-oxo-2-(4-hydroxyphenyl)ethyl)-D(or L)-xylose
2-deoxy-2-(2-oxo-2-(4-hydroxyphenyl)ethyl)-D(or L)-fucose
2-deoxy-2-(2-oxo-2-(4-hydroxyphenyl)ethyl)-D(or L)-lactose
2-deoxy-2-(2-oxo-2-(4-hydroxyphenyl)ethyl)-D(or L)-rhamnose
2-deoxy-2-(2-oxo-2-(4-methoxyphenyl)ethyl)-D(or L)-glucose
2-deoxy-2-(2-oxo-2-(4-methoxyphenyl)ethyl)-D(or L)-galactose
2-deoxy-2-(2-oxo-2-(4-methoxyphenyl)ethyl)-D(or L)-maltose
2-deoxy-2-(2-oxo-2-(4-methoxyphenyl)ethyl)-D(or L)-mannose
2-deoxy-2-(2-oxo-2-(4-methoxyphenyl)ethyl)-D(or L)-xylose
2-deoxy-2-(2-oxo-2-(4-methoxyphenyl)ethyl)-D(or L)-fucose
2-deoxy-2-(2-oxo-2-(4-methoxyphenyl)ethyl)-D(or L)-lactose
2-deoxy-2-(2-oxo-2-(4-methoxyphenyl)ethyl)-D(or L)-rhamnose
2-deoxy-2-(2-hydroxy-2-(3-methoxy-4-hydroxyphenyl)ethyl)-D(or L)-glucose
2-deoxy-2-(2-hydroxy-2-(3-methoxy-4-hydroxyphenyl)ethyl)-D(or L)-galactose
2-deoxy-2-(2-hydroxy-2-(3-methoxy-4-hydroxyphenyl)ethyl)-D(or L)-maltose
2-deoxy-2-(2-hydroxy-2-(3-methoxy-4-hydroxyphenyl)ethyl)-D(or L)-mannose
2-deoxy-2-(2-hydroxy-2-(3-methoxy-4-hydroxyphenyl)ethyl)-D(or L)-xylose
2-deoxy-2-(2-hydroxy-2-(3-methoxy-4-hydroxyphenyl)ethyl)-D(or L)-fucose
2-deoxy-2-(2-hydroxy-2-(3-methoxy-4-hydroxyphenyl)ethyl)-D(or L)-lactose
2-deoxy-2-(2-hydroxy-2-(3-methoxy-4-hydroxyphenyl)ethyl)-D(or L)-rhamnose
2-deoxy-2-(2-(4-hydroxyphenyl)ethyl)-D(or L)-glucose
2-deoxy-2-(2-(4-hydroxyphenyl)ethyl)-D(or L)-galactose
2-deoxy-2-(2-(4-hydroxyphenyl)ethyl)-D(or L)-maltose
2-deoxy-2-(2-(4-hydroxyphenyl)ethyl)-D(or L)-mannose
2-deoxy-2-(2-(4-hydroxyphenyl)ethyl)-D(or L)-xylose
2-deoxy-2-(2-(4-hydroxyphenyl)ethyl)-D(or L)-fucose
2-deoxy-2-(2-(4-hydroxyphenyl)ethyl)-D(or L)-lactose
2-deoxy-2-(2-(4-hydroxyphenyl)ethyl)-D(or L)-rhamnose
2-deoxy-2-(2-methylamino-2-(4-hydroxyphenyl)ethyl)-D(or L)-glucose
2-deoxy-2-(2-methylamino-2-(4-hydroxyphenyl)ethyl)-D(or L)-galactose
2-deoxy-2-(2-methylamino-2-(4-hydroxyphenyl)ethyl)-D(or L)-maltose
2-deoxy-2-(2-methylamino-2-(4-hydroxyphenyl)ethyl)-D(or L)-rhamnose
2-deoxy-2-(2-methylamino-2-(4-hydroxyphenyl)ethyl)-D(or L)-xylose
2-deoxy-2-(2-methylamino-2-(4-hydroxyphenyl)ethyl)-D(or L)-fucose
2-deoxy-2-(2-methylamino-2-(4-hydroxyphenyl)ethyl)-D(or L)-lactose
2-deoxy-2-(2-methylamino-2-(4-hydroxyphenyl)ethyl)-D(or L)-rhamnose
2-deoxy-2-(2-amino-2-(4-hydroxyphenyl)ethyl)-D(or L)-glucose
2-deoxy-2-(2-amino-2-(4-hydroxyphenyl)ethyl)-D(or L)-galactose
2-deoxy-2-(2-amino-2-(4-hydroxyphenyl)ethyl)-D(or L)-maltose
2-deoxy-2-(2-amino-2-(4-hydroxyphenyl)ethyl)-D(or L)-mannose
2-deoxy-2-(2-amino-2-(4-hydroxyphenyl)ethyl)-D(or L)-xylose
2-deoxy-2-(2-amino-2-(4-hydroxyphenyl)ethyl)-D(or L)-fucose
2-deoxy-2-(2-amino-2-(4-hydroxyphenyl)ethyl)-D(or L)-lactose
2-deoxy-2-(2-amino-2-(4-hydroxyphenyl)ethyl)-D(or L)-rhamnose
2-deoxy-2-(2-hydroxy-2-(4-hydroxyphenyl)ethyl)-D(or L)-lyxose
2-deoxy-2-(2-hydroxy-2-(4-methoxyphenyl)ethyl)-D(or L)-lyxose
2-deoxy-2-(2-oxo-2-(4-hydroxyphenyl)ethyl)-D(or L)-lyxose
2-deoxy-2-(2-oxo-2-(4-methoxyphenyl)ethyl)-D(or L)-lyxose
2-deoxy-2-(2-hydroxy-2-(3-methoxy-4-hydroxyphenyl)ethyl)-D(or L)-lyxose
2-deoxy-2-(2-(4-hydroxyphenyl)ethyl)-D(or L)-lyxose
2-deoxy-2-(2-methylamino-2-(4-hydroxyphenyl)ethyl)-D(or L)-lyxose
2-deoxy-2-(2-amino-2-(4-hydroxyphenyl)ethyl)-D(or L)-lyxose
2-deoxy-2-(2-hydroxy-2-(4-hydroxyphenyl)ethyl)-D(or L)-arabinose
2-deoxy-2-(2-hydroxy-2-(4-methoxyphenyl)ethyl)-D(or L)-arabinose
2-deoxy-2-(2-oxo-2-(4-hydroxyphenyl)ethyl)-D(or L)-arabinose
2-deoxy-2-(2-oxo-2-(4-methoxyphenyl)ethyl)-D(or L)-arabinose
2-deoxy-2-(2-hydroxy-2-(3-methoxy-4-hydroxyphenyl)ethyl)-D(or L)-arabinose
2-deoxy-2-(2-(4-hydroxyphenyl)ethyl)-D(or L)-arabinose
2-deoxy-2-(2-methylamino-2-(4-hydroxyphenyl)ethyl)-D(or L)-arabinose
2-deoxy-2-(2-amino-2-(4-hydroxyphenyl)ethyl)-D(or L)-arabinose
2-deoxy-2-(2-hydroxy-2-(4-hydroxyphenyl)ethyl)-D(or L)-N-acetylglucosamine
2-deoxy-2-(2-hydroxy-2-(4-methoxyphenyl)ethyl)-D(or L)-N-acetylglucosamine
2-deoxy-2-(2-oxo-2-(4-hydroxyphenyl)ethyl)-D(or L)-N-acetylglucosamine
2-deoxy-2-(2-oxo-2-(4-methoxyphenyl)ethyl)-D(or L)-N-acetylglucosamine
2-deoxy-2-(2-hydroxy-2-(3-methoxy-4-hydroxyphenyl)ethyl)-D(or L)-N-acetylglucosamine
2-deoxy-2-(2-(4-hydroxyphenyl)ethyl)-D(or L)-N-acetylglucosamine
2-deoxy-2-(2-methylamino-2-(4-hydroxyphenyl)ethyl)-D(or L)-N-acetylglucosamine
2-deoxy-2-(2-amino-2-(4-hydroxyphenyl)ethyl)-D(or L)-N-acetylglucosamine
2-deoxy-2-(2-hydroxy-2-(4-hydroxyphenyl)ethyl)-D(or L)-N-acetylgalactosamine
2-deoxy-2-(2-hydroxy-2-(4-methoxyphenyl)ethyl)-D(or L)-N-acetylgalactosamine
2-deoxy-2-(2-oxo-2-(4-hydroxyphenyl)ethyl)-D(or L)-N-acetylgalactosamine
2-deoxy-2-(2-oxo-2-(4-methoxyphenyl)ethyl)-D(or L)-N-acetylgalactosamine
2-deoxy-2-(2-hydroxy-2-(3-methoxy-4-hydroxyphenyl)ethyl)-D(or L)-N-acetylgalactosamine
2-deoxy-2-(2-(4-hydroxyphenyl)ethyl)-D(or L)-N-acetylgalactosamine
2-deoxy-2-(2-methylamino-2-(4-hydroxyphenyl)ethyl)-D(or L)-N-acetylgalactosamine
2-deoxy-2-(2-amino-2-(4-hydroxyphenyl)ethyl)-D(or L)-N-acetylgalactosamine.

27. The C-Glycoside compound according to claim 26, wherein the compound of formula (I′) is selected from the group consisting of:
2-deoxy-2-(2-oxo-2-(4-hydroxyphenyl)ethyl)-D(or L)-glucose
2-deoxy-2-(2-hydroxy-2-(4-hydroxyphenyl)ethyl)-D(or L)-glucose
2-deoxy-2-(2-oxo-2-(4-methoxyphenyl)ethyl)-D(or L)-glucose
2-deoxy-2-(2-hydroxy-2-(4-methoxyphenyl)ethyl)-D(or L)-glucose.
